(19) United States
(12) Patent Application Publication
(10) Pub. No.: US 2023/0314454 A1
(43) Pub. Date: Oct. 5, 2023

(54) BLOOD COAGULATION TEST REAGENT, AND BLOOD COAGULATION TEST METHOD

(71) Applicant: Thrombo Translational Research Lab Inc., Kumamoto-shi (JP)

(72) Inventor: Yuichi KAMIKUBO, Kumamoto-shi, Kumamoto (JP)

(21) Appl. No.: 18/329,833

(22) Filed: Jun. 6, 2023

Related U.S. Application Data
(63) Continuation of application No. PCT/JP2021/030430, filed on Aug. 19, 2021.

(30) Foreign Application Priority Data
Dec. 7, 2020 (JP) 2020-202843

(51) Int. Cl. G01N 33/86; C07K 14/745

(52) U.S. Cl. CPC G01N 33/86; C07K 14/745; G01N 2333/974; G01N 2333/96452; G01N 2333/7454

(57) ABSTRACT

A highly sensitive blood coagulation test reagent that enables determining and evaluating bleeding risk of direct oral anticoagulants (DOACs) and the like accurately in hemorrhagic diseases, and the like are provided. A blood coagulation test reagent containing: an active form of a blood coagulation factor XI (FXIa); and tissue factor (TF). A blood coagulation test method including a step of measuring the amount of thrombin generated by reacting a biological sample as a test specimen with a test reagent containing: 0.5 pM to 1,000 pM of an active form of a blood coagulation factor XI(FXIa); and 1 fM to 1,000 fM of tissue factor.

### CROSS-REFERENCE TO RELATED APPLICATIONS

This application is a continuation of PCT application No. PCT/JP2021/030430 filed on Aug. 19, 2021, which is based on and claims priority to Japanese Patent Applications No. 2020-202843 filed on Dec. 7, 2020, the disclosures of which are incorporated herein by reference.

### TECHNICAL FIELD

The present invention relates to a blood coagulation test reagent. The present invention also relates to a blood coagulation test method.

### BACKGROUND ART

Thrombus plays an important host-defense function of preventing blood from flowing from the inside of the blood vessel to the outside of the blood vessel(hemostasis). In thrombus formation, thrombin, one of blood coagulation factors, converts fibrinogen, a fibrous blood coagulation factor, into fibrin, and activates platelets.

Thrombin is formed by blood coagulation cascade reactions involving blood coagulation factors with enzyme activities that are mainly composed of two different reaction phases. One is the initiation phase for forming a minute amount of thrombin, and the other is the amplification phase necessary for forming a large amount of thrombin (referred to as a thrombin burst) (FIG. 1). It is thought that a minute amount of thrombin formed in the initiation phase causes the subsequent thrombin amplification phase by feedback.

In the thrombin initiation phase, the active form of blood coagulation factor (F) VII (FVIIa) forms a complex with tissue factor (TF) to activate the blood coagulation FX and produce the active form of FX (FXa). The generated FXa binds to the active form of blood coagulation FV (FVa) to form prothrombinase complex, and the complex finally produces thrombin by its activation of prothrombin. However, this thrombin production is regulated by tissue factor pathway inhibitor (TFPI), which is an inhibitor specific for the FVIIa-TF complex.

To escape from TFPI control and produce minute amounts of thrombin required for triggering the amplification phase, activation of FX by the active forms of blood coagulation FIX (FIXa) and FVIII (FVIIIa) is crucial. FIXa required in the reaction is produced by FVIIa-TF activation of FIX. Meanwhile, the activation mechanism of FVIIIa remained unknown for a long time.

However, from the recent study it has been unveiled that the FXa product by TF-FVIIa still bound to the FVIIa-TF complex, that is, the trimer of FXa-FVIIa-TF, activates FVIII (Non-Patent Literature 1).

Thrombosis is a disease in which thrombin is excessively in the blood vessel, causing thrombi to completely occlude the vessel and block blood flow. Today, thrombosis is one of the most serious diseases as represented by myocardial infarction, stroke, and the like, and in fact approximately 15 million people die from thrombosis per year worldwide (statistical data as of 2016 according to World Health Organization). The number thereof is expected to increase further with the rapid aging of the population.

Thrombosis is treated mainly with anticoagulants that prevent excessive thrombin formation by suppressing blood coagulation. In recent years, direct oral anticoagulants (DOACs) targeting FXa that inhibit thrombin formation have been developed and widely used for treating patients with stroke and the like. In the case of conventional treatment using warfarin or heparin, the effect of the drug has been monitored by blood coagulation tests after dosing to minimize the appearance of side effects such as hemorrhage and the recurrence of thrombosis, and the dose thereof has been optimized based on the obtained test results.

In fact, in treatment with warfarin and heparin, a prothrombin time test (PT-INR) and an activated partial thromboplastin time test (APTT)are employed, respectively. Meanwhile, in the cases of DOACs, no monitoring test was carried out initially because the drug with a short half-life in blood after administration was easily controlled, and was considered as a safe anticoagulant with few side effects.

However, as the number of patients taking DOACs has increased, many cases of excessive bleeding after dosing have been reported, mainly in the elderly and patients with renal dysfunction. Such bleeding not only reduces the quality of life of the patients and, in the worst case, leads to death, but also burdens the patients with much cost medically and economically. The development of safer DOAC therapy with reducing bleeding risk is an urgent issue.

As for antithrombotic therapy, for example, Patent Literature 1 discloses the highly sensitive and rapid assay for measuring thrombin (TG) generated in a blood sample, wherein the blood sample is incubated with tissue factor (TF), FIXa, and CaCl₂ for 5 minutes; and measuring TG in the blood sample using H-D-cyclohexyl-arginyl-alanyl-arginyl-amide-methylcoumarin (AMC) and/or butyloxycarbonyl-valyl-prolinyl-arginyl-AMC (V-P-R-AMC).

### CITATION LIST

### Patent Literature

Patent Literature 1: Japanese Translation of PCT International Application Publication No. 2019-521324

### Non Patent Literature

Non Patent Literature 1: Blood. 130(14):1661-1670, 2017

### SUMMARY OF INVENTION

### Technical Problem

In the current treatment guideline, patients with the same thrombosis are treated with the same dose of DOACs. Accordingly, differences in the sensitivity and the reactivity of the patient to the drug are not taken into account. In bleeding patients, it is possible that due to too much effectiveness from the drug the blood coagulation activity may decrease markedly, and the hemostasis function may be lost.

Thus, even in anti-coagulant therapy with DOACs, personalized treatment, in which the effect of the drug is monitored and the dose of the drug is optimized for each patient, has been desired. However, since conventional blood coagulation tests such as prothrombin time (PT-INR) and activated partial thromboplastin time (APTT) cannot assess the anti-coagulant effect of DOACs accurately due to low detection sensitivity, the personalized treatment has not been achieved yet. Under such circumstances, the present inventors have earnestly examined a highly sensitive blood coagulation test method that is suitable for determining and evaluating the bleeding risk accurately, in treating various bleeding disorders.

An object of the present invention is to provide a new blood coagulation test reagent and a new blood coagulation test method.

### Solution to Problem

The present inventors have earnestly repeated investigation to solve the above-mentioned problem, consequently found that the following inventions suit the above object, and completed the present invention. That is, the present invention relates to the following inventions.

<1> A blood coagulation test reagent comprising: an active form of a blood coagulation factor XI (FXIa); and tissue factor (TF).
<2> The blood coagulation test reagent according to the <1>, comprising: 0.5 pM to 1,000 pM of the active form of the blood coagulation factor XI; and 1 fM to 1,000 fM of the tissue factor, wherein a ratio of the active form of the blood coagulation factor XI to the tissue factor (active form of blood coagulation factor XI/tissue factor) is from 30 to 10,000.
<3> The blood coagulation test reagent according to the <1> or <2>, wherein the blood coagulation test reagent is for testing an indicator related to hemorrhagic diseases caused by a decrease in blood coagulant activity.
<4> A blood coagulation test method comprising a step of measuring an amount of thrombin generated by reacting a biological sample as a test specimen with a test reagent comprising: 0.5 pM to 1,000 pM of an active form of a blood coagulation factor XI; and 1 fM to 1,000 fM of tissue factor.

### Advantageous Effects of Invention

According to the present invention, blood coagulation can be tested. The present invention is also suitable for accurately determining and evaluating bleeding risk due to DOACs and the like.

### BRIEF DESCRIPTION OF DRAWINGS

FIG. 1 is a figure showing the blood coagulation cascade reaction mechanism.

FIG. 2 is a figure showing inhibitory effects of DOACs added into control plasma on the thrombin formation.

FIG. 3 is a figure showing inhibitory effects of apixaban added into plasma derived from patients on the thrombin formation.

FIGS. 4A to 4D show changes in thrombin formation in plasma after the apixaban ingestion over time.

FIGS. 5A to 5E show the relationship between the blood coagulation parameters of plasma obtained by blood collection at peak time after apixaban ingestion and bleeding events.

### DESCRIPTION OF EMBODIMENTS

Although embodiments of the present invention will be described in detail hereinafter, the explanations of the constituent requirements described below are examples (representative examples) of the embodiments of the present invention, and the present invention is not limited to the following contents unless the gist of the present invention is changed. When the expression “to” is used herein, the expression is used as an expression including the numerical values to the left and right thereof.

### [Blood Coagulation Test Reagent of the Present Invention]

A blood coagulation test reagent of the present invention contains the active form of blood coagulation FXI (FXIa) and tissue factor (TF). The blood coagulation test reagent of the present invention is referred to merely as a test reagent of the present invention hereinafter.

### [Blood Coagulation Test Method of the Present Invention]

A blood coagulation test method of the present invention comprises a step of measuring the amount of thrombin formed by reacting a biological sample as a test specimen with a test reagent containing: 0.5 pM to 1,000 pM of FXIa; and 1 fM to 1,000 fM of tissue factor. The test specimen or blood coagulation under reaction conditions can be tested from the amount of thrombin formed. The blood coagulation test method of the present invention is referred to merely as a test method of the present invention hereinafter.

The test method of the present invention can also be performed using the test reagent of the present invention in the present application, and the constitutions corresponding to the inventions can be mutually used in the present application.

The present application relates to a blood coagulation test reagent containing tissue factor, a blood coagulation initiator, and the active form of blood coagulation FXI (FXIa as the main component. The present application also relates to the blood coagulation test method performed in vitro using the test reagent.

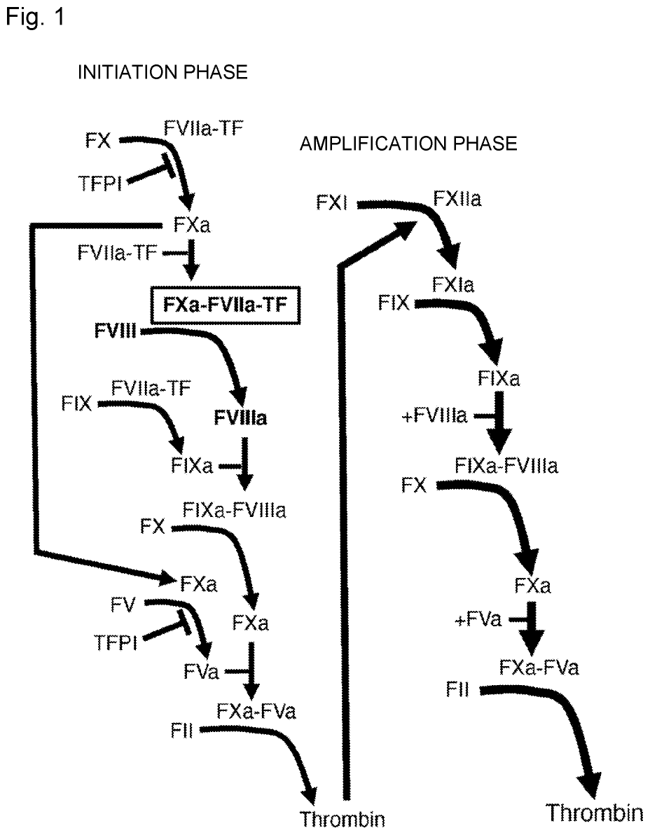

FIG. 1 is a figure showing the blood coagulation cascade reaction mechanism. The cascade reactions of blood coagulation comprises an initial phase for forming a minute amount of thrombin and an amplification phase necessary for forming a large amount of thrombin. Thrombin generated via FXa produced by TF-FVIIa activates FXI by feedback to amplify thrombin formation.

The present inventors have noticed the fact that the trimer of FXa-FVIIa-TF activates FVIII. When this is examined, it is considered that FXa formed through the action of FIXa-FVIIIa produces thrombin for causing the amplification phase. Thrombin produced in the initiation phase activates blood coagulation FXI by feedback to form its active form (FXIa). The formed FXIa enhances the amount of FIXa by converting FIX into the active form of FIXa. This further promotes the formation of FXa by FIXa-FVIIIa, enabling to amplify the thrombin formation finally. The present inventors notice this, and have performed examination and the like, and consequently created the blood coagulation test reagent containing TF and FXIa as the main components and the thrombin formation trial performed using the test agent. The present invention is based on the related findings.

The test reagent of the present invention and the test method of the present invention can provide a more highly sensitive test than when the active form of blood coagulation FIX (FIXa) is used. For this reason, the test can be performed utilizing small amount of the reagent. Since the active form of blood coagulation FXI and tissue factor are derived from the living body and difficultly produced on large scales, the present invention, in which test can be performed even with small amounts of the reagents, is useful. Since repeated tests and the like are also easily performed, the present invention is also suitable for an increase in the number of tests.

In addition, even in the case such as a therapy for lowering FIXa to prevent thrombosis or hemophilia B lacking FIX, where the test with FIXa is not suitable, the test according to the present invention is possible, and the present invention also contributes to the diversification of blood coagulation test techniques.

The test reagent of the present invention and the test method of the present invention can target mammals having FXIa in the blood coagulation reaction mechanisms. For example, human blood coagulation can be tested. Moreover, for example, the test reagent and the test method can target monkeys, dogs, cats, bovines, pigs, horses, rats, mice, guinea pigs, and the like. The test reagent and the test method can target mammalian pets, domestic animals, experimental animals, and the like.

The test reagent of the present invention and the test method of the present invention use the active form of blood coagulation FXI (FXIa), tissue factor, and the like. As these, FXIa, tissue factor, and the like derived from the same kind of organism as an organism at which the test is targeted may be used, or FXIa, tissue factor, and the like that are derived from another organism, and can be transferred to the test of the organism may be used. A substance that is identical with or 90% or more, 95% or more, 98% or more, or the like homologous to FXIa, has the function of FXIa, and is produced by genetic recombination or the like may be used. A substance that is identical with or 90% or more, 95% or more, 98% or more, or the like homologous to tissue factor, has the function of tissue factor, and is produced by genetic recombination or the like may be used.

[The Active Form of Blood Coagulation FXI (FXIa)]The test reagent of the present invention contains the active form of blood coagulation FXI (FXIa). In the present application, the activated active form of blood coagulation FXI may be described merely as “FXIa”.

In the test reagent of the present invention, the concentration of FXIa is preferably 0.5 pM to 1,000 pM. When the concentration is described in the present application, M may be described as an abbreviation for a concentration that is ·mol/L. mM is an abbreviation for m (10⁻³) ·mol/L. μM is an abbreviation for μ (10⁻⁶) ·mol/L. pM is an abbreviation for p (10⁻¹²) ·mol/L. fM is an abbreviation for f (10⁻¹⁵) ·mol/L.

If the test reagent FXIa is less than 0.1 pM, enough reaction may not occur in the blood coagulation test, and when trying to measure the amount of thrombin, results may be below the detection limit and may not be able to be evaluated. Also, even if the test reagent FXIa exceeds 1,000 pM, the amount of thrombin may exceed the amount required for the test and the reaction may be saturated.

The lower limit of FXIa of the test reagent is preferably 0.5 pM or more, and more preferable 1 pM or more. As the concentration of the test reagent is higher, the amount of the test reagent becomes suitable as a necessary amount for thrombin formation, enabling to perform a stable test.

The upper limit of the FXIa of the test reagent is preferably 500 pM or less, more preferably 200 pM or less, and further preferably 100 pM or less. If the amount necessary for the test is sufficient, FXIa may therefore be used according to the test conditions, and an excessive raw material may not be used.

### [Tissue Actor (TF)]

The test reagent of the present invention contains tissue factor (TF). In the present application, tissue factor may be described merely as “TF”.

In the test reagent of the present invention, the concentration of tissue factor is preferably 1 fM to 1,000 fM. If the test reagent TF is less than 1 fM, enough reaction may not occur in the blood coagulation test, and when trying to measure the amount of thrombin, results may be below the detection limit and may not be able to be evaluated. Also, even if the test reagent TF is more than 1,000 fM, the amount of thrombin may exceed the amount required for the test and the reaction may be saturated.

The lower limit of the TF of the test reagent is preferably 2 fM or more, and more preferable 3 fM or more. As the concentration of the test reagent is higher, the amount of the test reagent becomes suitable as a necessary amount for thrombin formation, enabling to perform the stable test.

The upper limit of the TF of the test reagent is preferably 500 fM or less, more preferably 200 fM or less, and further preferably 100 fM or less. If the amount necessary for the test is sufficient, TF may therefore be used according to the test conditions, and an excessive raw material may not be used.

### [Active Blood Coagulation FXI/Tissue Factor (FXIa/TF)]

In the test reagent of the present invention, the ratio of active blood coagulation FXIa to tissue factor (active blood coagulation FXI/tissue factor) is preferably 30 to 10,000. The “ratio of active blood coagulation FXI/tissue factor” may be described merely as the “FXIa/TF”. The FXIa/TF is a ratio between the molar concentrations in the test reagent. When the FXIa/TF is in the above-mentioned range, the amounts of the components necessary for test is suitable amounts, and a test can be stably performed. The FXIa/TF is more preferably 60 to 5,000, and further preferably 150 to 2,000.

The test reagent of the present invention can contain components other than FXIa and TF. The test reagent is usually used for the test in which the reaction is caused in a field containing water as the main component. Therefore, the medium of the test reagent can contain water as the main component. The test reagent can contain an adsorption inhibitor or a protective agent for proteins such as FXIa and TF, a pH adjustor, a mineral adjustor, and the like. For example, the test reagent can contain serum albumin and a buffer solution, synthetic phospholipids, calcium chloride, and the like.

The test method of the present invention can be performed using the test reagent of the present invention. Blood coagulation can be tested by the measurement of the amount of thrombin formed by reacting the test reagent of the present invention with a biological sample. It can be confirmed that if the amount of thrombin is small, blood hardly coagulates, and if the amount of thrombin is large, blood easily coagulates. When these tests are performed, the degree of blood coagulation when another test object is used in combination can be evaluated in vitro by making the test object coexist.

The test method of the present invention has a step of reacting the biological sample with the test reagent. The measurement of the amount of thrombin formed by this reaction enables testing blood coagulation.

This test can be used for testing the indices to various hemorrhagic diseases caused by a decrease in blood coagulation activity. The biological sample such as blood, plasma, urine, or the like can be targeted as a test specimen.

When the test method of the present invention is performed, the test reagent containing the active form of blood coagulation FXI (FXIa) in the concentration range of 0.5 pM to 1,000 pM and tissue factor (TF) in the concentration range of 1 fM to 1,000 fM can be used.

In this step, the biological sample and the test reagent as the thrombin formation initiation reagent are mixed, and shaken or left to stand for a fixed period of time for the reaction. The test specimen containing a test object, a component or a reagent used for controlling reaction conditions, and the like besides the biological sample and the test reagent may be used.

The temperature during the reaction can be adjusted to around 25 to 45° C., at which the reaction of these protein and the like occurs, and is preferably 30 to 40° C., further preferably 35 to 40° C., and particularly preferably 37 to 38° C. The reaction time can be adjusted to around 1 minute to 30 minutes, and can be adjusted to around 1 to 15 minutes, or around 2 to 5 minutes. The reaction can be stopped by mixing various reaction stopping chemicals. Examples of the reaction stopping chemicals include EDTA (ethylenediaminetetraacetic acid).

In the reaction thrombin is formed depending on the properties of the biological sample and the reaction conditions. In the reactions the test can be performed with reference to Patent Literature 1 (Japanese Translation of PCT International Application Publication No. 2019-521324) according to the assay thereof using the active form of blood coagulation FXI (FXIa) instead of the active form of blood coagulation FIX (FIXa). Since FXIa is more highly reactive than FIXa, the FXIa concentration can be adjusted to a concentration such that the ratio of the FXIa concentration to the FIXa concentration in Patent Literature 1 (FXIa/FIXa) is 1/10 to 1/100 or 1/20 to 1/50.

The blood coagulation test according to the present invention can be performed for the purpose of supporting the prevention and the treatment of inherent or acquired hemorrhagic disease (hemorrhage). For example, a bleeding risk due to the medication is determined and evaluated by performing the test in the treatment of thrombosis (strike, myocardial infarction, or the like) using antithrombotic drugs; and the test is performed to achieve safe treatment in which excessive bleeding is prevented by optimizing the type and the dose of a drug for each patient.

The test can also be used for screening patients with inherent hemorrhagic diseases represented by hemophilia and for determining the effects of therapeutic drugs on those patients.

Furthermore, the test is also very useful in the determination of bleeding risk caused by DOACs. The thrombin generation test is an assay method for determining blood coagulation activity by quantifying thrombin generated after adding the thrombin generation reagent containing calcium into blood or plasma that is a test specimen, followed by incubation for a fixed period of time.

Since prothrombinase complex generates thrombin, this test is a test method for measuring the amount of the prothrombinase complex activity in blood. Although test reagents and test methods for thrombin generation test have been examined until now, coagulation test that enables evaluating and determining bleeding risk caused by DOACs have not been developed.

The present inventors have invented the innovative thrombin generation test having new performance by improving the test reagent used for the thrombin generation test. It is considered that the thrombin generation test contributes to the achievement of a safer personalized therapy, in which side effect is reduced in treatment with DOACs.

The invention according to the present application can be grasped as follows.
A marker for determining disease and the like containing the active form of blood coagulation FXI (FXIa) and tissue factor (TF).
A method for determining disease and the like having a step of measuring the amount of thrombin formed by reacting a biological sample as a test specimen with the test reagent containing the active form of blood coagulation FXI and tissue factor.
A method for evaluating the possibility that a patient contracts a disease and the like by determining the amount of thrombin formed by reacting a biological sample as a test specimen with the test reagent containing the active form of blood coagulation FXI and tissue factor.
A mixture of an activated the active form of blood coagulation FXI and tissue factor to be used as a diagnostic marker for disease or the like.
A method for collecting data for diagnosing a disease or the like, wherein the amount of thrombin formed by reacting a biological sample as a test specimen with the test reagent containing the active form of blood coagulation FXI and tissue factor is measured.
An assay method for evaluating the possibility of suffering from a disease or the like by comparing the concentration of thrombin formed by reacting a biological sample as a test specimen with the test reagent containing the active form of blood coagulation FXI and tissue factor with the concentration in the specimen of a healthy person.
An in vitro method for assisting in the detection of a disease or the like in a test subject, comprising the step of detecting the concentration of thrombin formed by reacting a biological sample as a test specimen with the test reagent containing the active form of blood coagulation FXI and tissue factor.

As these diseases and the like, various diseases related to blood coagulation can be targeted according to this application as described above,

### EXAMPLES

It is proved through the Examples in the clinical performance study using the following patient plasma specimens that the thrombin generation test is useful in the determination of bleeding risk caused by DOACs medication. The present invention is not limited to the following Examples unless the gist thereof is changed.

### 1 Method

### 1) Target Patients in the Clinical Performance Study and Preparation of Plasma Specimens

After the approval of the ethical review committee of Health Sciences University of Hokkaido, the clinical performance test was conducted in patients diagnosed as non-valvular atrial fibrillation who were undertaking treatment with DOACs for preventing cardiogenic cerebral embolism. When blood specimens were collected, the attending physician gave oral and written explanation to the patients. After obtaining the consent form with the signature, blood was collected. Hemorrhage that occurred in relation to DOAC medication was defined according to hemorrhage criteria of the International Society on Thrombosis and Haemostasis and ROCKET AF, and patients meeting the criteria were considered to be hemorrhagic patients.

In test, plasma specimens were prepared by centrifugation of blood that was collected using an anticoagulant citric acid. The plasma specimens were frozen at −80° C. until the test starts, and thawed at 37° C. immediately before the test, and used.

### 2) Assay of FIXa-FVIIIa-Dependent Thrombin Generation

Assay of FIXa-FVIIIa-dependent thrombin generation was performed in the following (1) to (3).

### (1) Preparation of the Thrombin Generation Initiation Reagent Using in the Thrombin Generation Test

The thrombin generation initiation reagent was prepared by mixing the following reagents. The thrombin formation initiation reagent was appropriately diluted with 50 mM tris buffer solution containing 0.5% bovine serum albumin (purchased from FUJIFILM Wako Pure Chemical Corporation) and 0.15 M sodium chloride (purchased from FUJIFILM Wako Pure Chemical Corporation) (BSA/TBS, pH 7.4).
Human TF (Dade Innovin, purchased from SYSMEX CORPORATION) solution
Human FXIa (purchased from Funakoshi Co., Ltd.) solution
Synthetic phospholipids (purchased from Haematex) solution
Calcium chloride (purchased from FUJIFILM Wako Pure Chemical Corporation) solution

### (2) Preparation of the Thrombin Detection Reagent

A fluorogenic thrombin substrate (Pefafluor TH: D-cyclohexylalanine-alanine-arginine-amido-methylcoumarin, from DSM Nutritional Products) solution and ethylenediaminetetraacetic acid (EDTA, purchased from Tokyo Chemical Industry Co., Ltd.) solution were mixed to prepare the thrombin detection reagent.

As representative Example, a mixture of 50 μM thrombin fluorogenic substrate and 10 mM EDTA were used.

### (3) Trial Method

First, test plasma specimens were dispensed to wells of a 96-well microtiter plate (purchased from Thermo Fisher Scientific K.K.). In the first reaction, the thrombin generation initiation reagent was added to the plasma specimens, followed by incubation at 37° C. for 2.5 minutes to generate thrombin.

To quantify the generated thrombin, the thrombin detection reagent containing EDTA, which was a reaction stopping reagent, was then added to the plasma, followed by incubation at 37° C. for 1 minute. Since thrombin hydrolyzed the thrombin fluorogenic substrate to emit fluorescence during the incubation, the fluorescence intensity was measured using a fluorescence plate reader (excitation wavelength: 355 nm, fluorescence wavelength: 460 nm). The measured fluorescence intensity was converted into the thrombin concentration based on a standard curve constructed using a thrombin standard substance (Technothrombin TGA calibrator, purchased from Cosmo Bio Co., Ltd.). The thrombin generation in the plasma was expressed as a relative ratio (%) with respect to the thrombin generation in control plasma (Siemens control plasma N for blood coagulation test, purchased from SYSMEX CORPORATION).

### 3) Blood Coagulation Test

The coagulant activity of each of the test plasma specimen was tested using APTT and PT-INR test methods as blood coagulation tests. In both methods, the coagulant activity was determined by measuring clotting time after adding the coagulation initiation reagent into plasma specimens. The DOAC concentration in the plasma was determined based on its inhibitory activity toward the activity of FXa added into the plasma.

### 4) Thrombin Generation in Plasma to Which DOACs were Added: DOAC Spiking Test

To test inhibitory effect of the DOACs on thrombin generation, Test specimens were prepared by spiking rivaroxaban, apixaban, or edoxaban into plasma, and the thrombin generation in the plasma was assessed.

### 5) Statistical Analysis

Statistical analysis of the significant difference between two groups was performed using Graphpad Prism 8, which was statistical analysis software (purchased from GraphPad Software).

### 2 Results

### 1) Example 1: Influence of DOACs on Blood Coagulation Activity of Control Plasma

Three DOACs (rivaroxaban, apixaban, and edoxaban) were each added to the control plasma in the concentration range of 18.5 ng/mL to 500 ng/mL for testing the effect of DOACs on the blood coagulant activity of the control plasma, and the thrombin generation was examined.

Thrombin was generated by adding the initiation reagent containing 50 fM TF, 1.6 pM FXIa, and 20 μM synthetic phospholipids into the plasma concomitantly with 6.8 mM calcium chloride. For determination of the mixing ratio of the plasma to the initiation reagent, the thrombin generation test was performed by the method, in which 10 μL of the initiation reagent was added into 35 μL of the plasma, or the thrombin generation was performed by a method, in which 60 μL of the initiation reagent was added into 20 μL of the plasma if the plasma needed to be more diluted for the test. The concentration ranges of the DOACs used for the test is almost the same as the ranges of the DOAC concentrations detected in plasmas after administration of the drugs into patients.

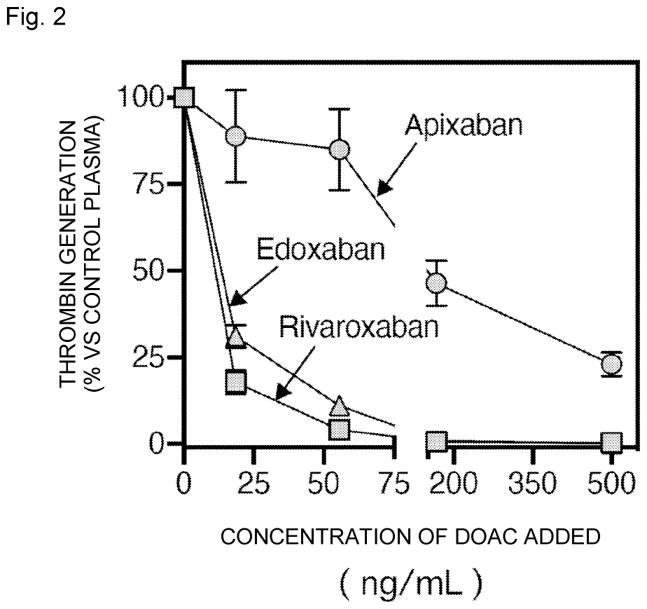

The three DOACs dose-dependently suppressed the thrombin generation. Especially, rivaroxaban had the strongest inhibitory effect (FIG. 2). The results revealed that the thrombin generation test enabled detecting inhibitory effect of the DOACs on blood coagulation with high sensitivity.

FIG. 2 is a figure showing the inhibitory effects of DOACs added into control plasma on the thrombin generation. Three DOACs (rivaroxaban, apixaban, and edoxaban) were added into the plasma in the concentration range of 18.5 ng/mL to 500 ng/mL for examining the effect of DOACs on the thrombin generation in the control plasma, and the thrombin generation was assessed.

An initiation reagent containing 50 fM TF, 1.6 pM FXIa, 20 μM synthetic phospholipids, and 6.8 mM calcium chloride was added into the plasma to generate thrombin. The values in the figure show the average values and the standard deviation values of two experiments.

### 2) Example 2: Investigation of Individual Variation in Inhibitory Effect of DOAC on Thrombin Generation

To investigate the individual variation in inhibitory effect of DOAC on the thrombin generation, apixaban was added into each of plasma obtained from three patients (ID#: 001, 002, and 003) at a concentration of 125, 250, or 500 ng/mL, and the inhibitory effect of apixaban on the thrombin generation in the plasma was examined.

The initiation reagent containing 150 fM TF, 12.5 pM FXIa, and 20 μM synthetic phospholipids was added concomitantly with 16 mM calcium chloride to form thrombin.

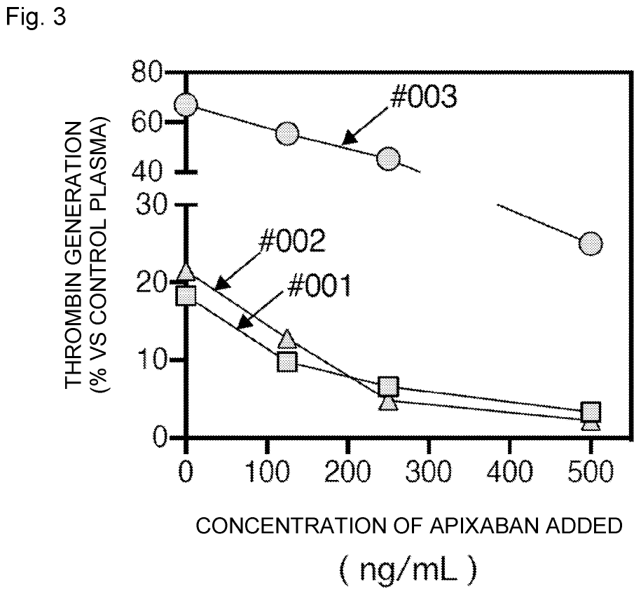

Blood was collected from the patient before taking apixaban and then centrifuged to prepare plasma. The addition of apixaban suppressed thrombin generation in the plasma from the patient in a dose-dependent fashion, and the individual variation in the inhibitory effects was observed. In fact, the thrombin generation in the patient #001 and #002 decreased to 5% or less of that of the control plasma by adding apixaban, while the thrombin generation was maintained at 25% in the patient #003 (FIG. 3). This suggests that there is the individual variation in the sensitivity and reactivity of the patients to the DOAC.

FIG. 3 is a figure showing the inhibitory effect of apixaban added into the plasmas derived from patients on the thrombin generation. To test the inhibitory effect of DOAC on thrombin generation in the patient plasmas, apixaban was added into each of the plasma at 125, 250, or 500 ng/mL, and the thrombin generation formation was examined. Blood was collected from three patients (ID#: 001, 002, and 003) before taking apixaban and then blood was centrifuged to prepare the patient plasma.

The initiation reagent comprising 150 fM TF, 12.5 pM FXIa, 20 4M synthetic phospholipids, and 16 mM calcium chloride was added to each of the plasma to form thrombin.

### 3) Example 3: The Time-Dependent Changes in Thrombin Generation in Plasma After Taking Apixaban

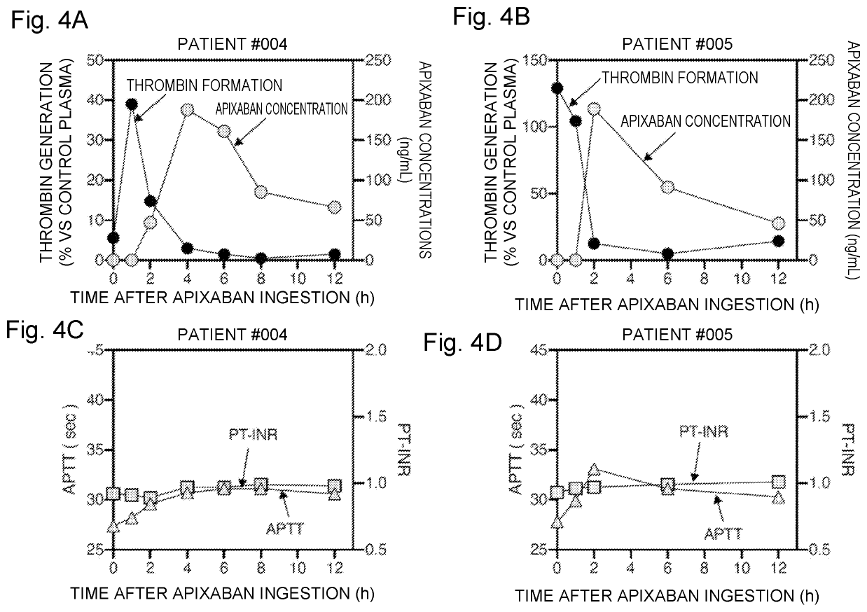

In two patients (ID#: 004 and 005) who took 10 mg of apixaban, blood was collected over time immediately after taking the drug, and the time-dependent changes in apixaban concentration, thrombin generation, and blood coagulant activities measured by APTT and PT-INR in the plasma were monitored. As a result, the apixaban concentrations peaked 2 to 4 hours after taking the drug, and then the concentrations decreased due to clearance from the blood (FIGS. 4A and 4C).

The thrombin generation decreased with increase in apixaban concentrations in the patients #004 (FIG. 4A) and #005 (FIG. 4C), and exhibited the lowest values between 2 hours and 4 hours after. Meanwhile, no significant alteration in the blood coagulant activities measured by APTT and PT-INR was observed after taking apixaban (FIGS. 4B and 4D). The results indicated that the thrombin generation test enabled evaluating the anticoagulant action of apixaban with higher sensitivity than the conventional coagulation tests.

FIGS. 4A to 4D show the changes in the thrombin generation in plasma over time after taking apixaban. In two patients (ID#: 004 and 005) who took 10 mg of apixaban, blood was collected over time immediately after taking the drug, and the time-dependent changes in the apixaban concentrations, the thrombin generation, and the blood coagulant activities measured by APTT and PT-INR in the plasma were monitored. As a mixing reagent for initiation of thrombin generation, the same reagent as in FIG. 3 was used.
A. Measured values of apixaban concentration and thrombin formation in plasma from patient #004
B. Blood coagulation activity values measured in plasma from patient #004 by APTT and PT-INR
C. Measured values of apixaban concentration and the thrombin generation in plasma from patient #005
D. Blood coagulant activity values measured in plasma from patient #005 by APTT and PT-INR

### 4) Example 4: The Relationship Between the Thrombin Generation in Plasma Prepared from Collected Blood at Time of Peaks After Taking Apixaban and Hemorrhagic Events

To test the relationship between the thrombin generation and the hemorrhagic events, patients who took apixaban were divided into the patients with the hemorrhagic events (the number of specimens: 6) and non-hemorrhagic patients (the number of specimens: 83), and the thrombin generation in the plasma was compared using the test reagent according to the present invention. As the mixing reagent for initiation of the thrombin generation, the same reagent as in FIG. 3 was used. In addition, the thrombin generation in the plasma was compared using the initiation mixture described in Japanese Translation of PCT International Application Publication No. 2019-521324 (150 fM TF, 100 pM FIXa, 20 μM synthetic phospholipids, and 16 mM calcium chloride). Besides, the apixaban concentrations and the coagulant activity values measured by APTT and PT-INR in the plasma were also compared in the same way. Blood was collected between 1 to 4 hours after taking apixaban (at the time of peaks) to prepare the plasma specimens.

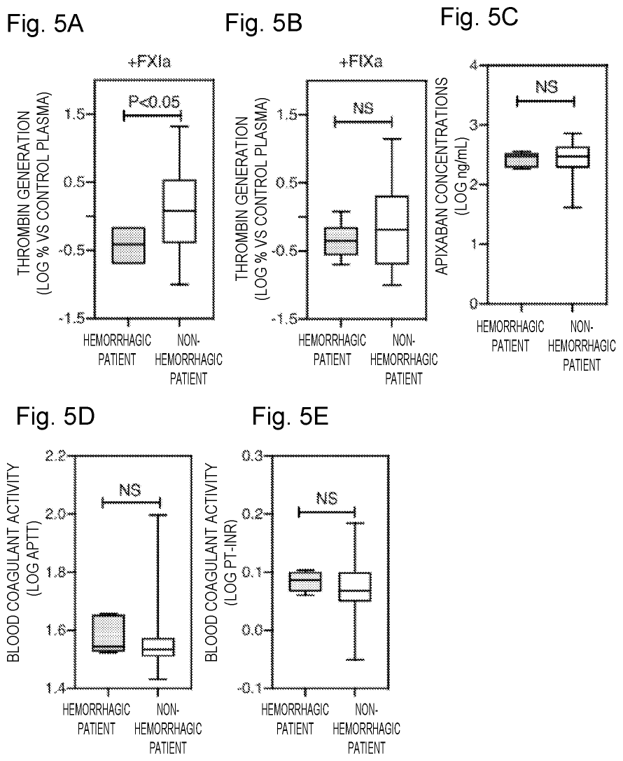

In the test results obtained by the present invention, the level of the thrombin generation in the plasma prepared from the patients with the hemorrhagic events were significantly low as compared with the non-hemorrhagic group (FIG. 5A; p<0.05 by Mann-Whitney statistical analysis). In contrast, no significant difference in the thrombin generation result (FIG. 5B) using TF and FIXa and described in Japanese Translation of PCT International Application Publication No. 2019-521324, the apixaban concentrations (FIG. 5C), and the coagulant activity values by APTT (FIG. 5D) and PT-INR (FIG. 5E) was observed between both groups. The above-mentioned results showed that only the thrombin generation test in the present invention enabled evaluating bleeding risk caused by apixaban.

FIGS. 5A to 5E show the relationship between the blood coagulation parameters in plasma obtained from collected blood at the time of peak after taking apixaban and the hemorrhagic events. The patients who took apixaban were divided into the patients with the hemorrhagic events (n=6) and the non-hemorrhagic patients (n=83), and the blood coagulation parameters in the plasma were compared. Blood was collected between 1 to 4 hours after taking apixaban (at the time of peaks) to prepare the plasma specimens. As a mixing reagent for initiation of the thrombin generation, the same reagent as in FIG. 3 was used.

A. Thrombin generation value by the present invention.
B. Thrombin generation value by the test using TF and FIXa and described in Japanese Translation of PCT International Application Publication No. 2019-521324.
C. Apixaban concentrations.
D. Blood coagulant activity value by the APTT.
E. Blood coagulant activity value by PT-INR.

The measured values were converted into logarithmic values, and the logarithmic values were plotted by the box and whisker plot, showing the medians, the minimums, and the maximums. The difference between two groups was determined by Mann-Whitney test, and p-value <0.05 was considered statistically significant. NS: not significant.

### INDUSTRIAL APPLICABILITY

The blood coagulation test reagent of the present invention can be utilized for testing blood coagulation, and has industrial applicability. The blood coagulation test method of the present invention is also a method for analyzing what is collected from humans to gather various data, but is not medical practice, which a doctor performs on a human, does not correspond to a method for operating, treating, or diagnosing a human, and has industrial applicability.

1. A blood coagulation test reagent comprising: an active form of a blood coagulation factor XI (FXIa); and tissue factor (TF).

2. The blood coagulation test reagent according to claim 1, comprising:
5 pM to 1,000 pM of the active form of the blood coagulation factor XI; and
1 fM to 1,000 fM of the tissue factor,
wherein a ratio of the active form of the blood coagulation factor XI to the tissue factor (active form of blood coagulation factor XI/tissue factor) is from 30 to 10,000.

3. The blood coagulation test reagent according to claim 1, wherein the blood coagulation test reagent is for testing an indicator related to hemorrhagic diseases caused by a decrease in blood coagulant activity.

4. A blood coagulation test method comprising a step of measuring an amount of thrombin generated by reacting a biological sample as a test specimen with a test reagent comprising: 0.5 pM to 1,000 pM of an active form of a blood coagulation factor XI; and 1 fM to 1,000 fM of tissue factor.
